(19) United States
(12) Patent Application Publication
(10) Pub. No.: US 2003/0236448 A1
(43) Pub. Date: Dec. 25, 2003

(54) Oxidase-base sensors for selective analysis of analytes in aqueous samples

(76) Inventors: Fang Lu, Millbrae, CA (US); Frank N. W. Lu, San Mateo, CA (US); Kai Hua Wang, San Bruno, CA (US)

Correspondence Address: John H. Faro, Esq., Faro & Associates, P.O. Box 4904, Key Biscayne, FL, 33149-4904, US

(21) Appl. No.: 09/818,000

(22) Filed: Mar. 27, 2001

(51) Int. Cl. A61B 5/00

(52) U.S. Cl. 600/300

(57) ABSTRACT

A system and method for the selective electrochemical analysis of an aqueous test sample for an analyte of interest, with biosensor comprising a working electrode comprising (a) an effective amount of oxidase enzyme specific for the analyte of interest, and (b) a metal catalysts doped carbon composition specific for the catalytic reduction of hydrogen peroxide liberated from enzymatic action of said oxidase upon the analyte of interest. The system and method of this invention effectively perform such selective electrochemical analysis in the presence of common interferents (e.g. acetaminophen, uric and ascorbic acids, catecholamines) at relative low voltages (from about +0.3 to −0.25V), so as to avoid generation of an overlapping signal from such interferents.

### BACKGROUND OF THE INVENTION

1. Field of the Invention

This invention relates to a method and to a device for the selective analysis of analytes in an aqueous sample by electrochemical means. More specifically, this invention is directed to test device for the selective amperometric measurement of hydrogen peroxide as indicative of the presence and concentration of an analyte of interest in an aqueous sample.

2. Description of the Prior Art

The use of so-called “biosensors” to directly detect the presence and concentration of an analyte of interest by amperometric and/or potentiometric measurement is well-known. The various electrochemical mechanisms that lend themselves to such direct detection techniques are also well-known. In brief, such techniques involve contacting a sample with a reagent (e.g. enzyme), under analytical conditions, to effect the release or formation of a compound that is electrochemically active, or can be converted to a compound or species that is electrochemically active. The electrochemically active compound has a characteristic signal that is manifest as a change in voltage and/or current can then be monitored, and the recorded signal correlated/compared with a standard response curve for determination of the presence and, possibly, the concentration of the analyte of interest.

One of the model systems typically used to exemplify such electrochemical analysis, comprises the quantitative determination of glucose in a biological sample. As is fully appreciated, the precision determination of quantitative glucose levels in biological fluid samples is critical to control of diabetes in both insulin dependent (Type I) diabetics and diabetics that are capable of control of their blood sugar by rigorous adherence to diet, Type II diabetics. The biological fluid specimen typically used in such analysis of glucose by such individuals can include venous blood, capillary blood or interstitial fluid. The biochemical reaction used to determine the amount of glucose in the sample typically involves either an a spectrophotometric method or an electrochemical method. Electrochemical analysis has been widely used in detection of glucose in biological sample because of its accuracy, convenience and cost over the spectrophotometric method.

Historically, the biosensors used in the electrochemical analysis of glucose include an enzyme electrode (e.g. glucose oxidase) which reacts with the glucose in the sample. More specifically, oxidase-based amperometric biosensors have traditionally relied upon the immobilization of an oxidase enzyme on the surface of various transducers and the detection of the current associated with the redox product in the biological reaction. The four basic approaches (e.g. mechanisms) that have been used in conjunction with amperometric biosensors for measurement of glucose concentration, include/involve (a) measuring oxygen consumption, (b) products generation, (c) dual enzyme configurations and (d) enzyme mediators.

(a) Measurement of consumption of the molecular oxygen—In this analytical regime, the consumption of oxygen (uptake) by the enzyme, glucose oxidase, is measured. Not only is the reaction kinetics involved in such uptake relatively slow, but also the results subject to error because of the ambient concentration of oxygen in the samples. Accordingly, this method is generally regarded as unreliable and the results obtained often inaccurate and not reproducible.

(b) Measurement of oxidation of hydrogen peroxide in sample—In this analytical regime, glucose determination is measured as a function of the current generated incident to the electrochemical oxidation of the hydrogen peroxide in the biological sample. Unfortunately, the electrochemical oxidation of hydrogen peroxide requires operation of the electrochemical process at a relatively a high oxidation potential. At such higher potentials, common interferent species in the test sample also undergo oxidation and thereby produce an overlapping signal. In order to effect selective measurement of the analyte of interest in a complex biomatrix, anti-interference membranes (e.g., cellulose acetate) are normally used to improve the sensor discrimination. However, even with this precaution, the sensor still suffers from residual interference effects which are particularly severe when acetaminophen is present in the sample.

(c) Measurement of glucose with multiple enzyme doped transducers—The use of amperometric biosensors having multi-enzyme reaction systems can improve transducer selectivity and sensitivity. This class of biosensor uses a lower operating potential of the second enzyme to eliminate the interference. In, for example, the electrochemical analysis of glucose, the enzyme electrode is doped with both horseradish peroxidase and glucose oxidase. This type of transducer can minimize some of the common interferences. However, because of the kinetics of the reaction cascade, (e.g. the need for enzymatic liberation of excess peroxide to drive the reaction in the desired direction), this approach is unduly complex and highly sensitive to enzyme activity and efficiency. Accordingly, it has not received widespread acceptance.

(d) Use of enzyme mediator to improve reproducibility in analysis—The replacement of the enzyme's natural electron acceptor by an artificial mediator is a good choice for oxidase-based amperometric biosensor. Usually the artificial mediator is a low molecular-weight species with the reversible heterogeneous kinetics and a low over-potential for regeneration. The function of the artificial mediators is to shuttle electrons between the enzyme and the electrode, and thereby eliminate the dependence upon the electroactive species and enhance the reproducibility. Because of the regeneration of the mediator at lower detecting potentials, oxidation of the potentially interferent compounds is significantly reduced. Notwithstanding such improvement in selectivity, mediated amperometric biosensors are still sensitive to changes in dioxygen tension and also suffer from the problem of the mediator leaching into the sample. Both of these limitations/deficiencies result in introduction of significant error in the determination of analyte in a test sample.

In each of the alternative electrochemical analysis regimes discussed above, additional operational steps and reagents are required; and, whatever improvement is accomplished in regard to sensitivity and selectivity was, at best marginal, because of the incomplete elimination of interfering signals.

In order to resolve the foregoing problems, a mediator-free and membrane-free biosensor was described for the first time in 1993 by, J. Wang, F. Lu, D. Lopez and H. Tobias, Anal. Letter., 26(6), 1819, (1993). The Wang & Lu biosensor was reportedly the first method to provide a means for measuring cathodic current of enzymatically liberated hydrogen peroxide in a carbon paste biosensors. The make-shift device used by Wang & Lu as their biosensor was prepared by filling one end of a Teflon tube with a mixture containing ruthenium on carbon (5% Ru) dispersed in mineral oil (40/60% w/w carbon/oil). After further processing of the resultant carbon paste, glucose oxidase was added to the mixture. Once the resultant paste was packed into one end of a Teflon tube, a copper wire was inserted in the other end thereof to complete the electrical connection. The foregoing device was effective to selectively determine the concentration of glucose in a biological fluid sample containing interfering substances. Notwithstanding this technical advance in the electrochemical analysis of glucose, the Wang & Lu biosensor was cumbersome to fabricate and did not readily lend itself to simplicity of manufacture or use.

Microparticle carbon based biosensors have also recently been disclosed wherein carbon microparticles are conjugated to peroxidase; the resultant conjugate dispersed within a polymer matrix; and, the matrix thereupon coated upon an electrode surface, so as to place the peroxidase in intimate contact with the electrode surface. Thus, upon the enzymatic liberation of hydrogen peroxide in proximity to the surface of the matrix on the electrode surface, the hydrogen peroxide selectively diffuses into the matrix. The peroxidase, thereafter, reduces the hydrogen peroxide within the matrix and generates a signal current of useable magnitude, even at relatively high concentrations of glucose in the sample, U.S. Pat. No. 5,755,953, (to Henning et al, issued May 26, 1998). The Henning et al., polymer matrix used in the coating of the microparticle/conjugate mixture, has the added property of a semi-permeable membrane, at least to the degree of exclusion of potential interferents from interaction with the peroxidase conjugate. The Henning et al., is thus dependent for its success upon the exclusion of interferents from the transducer so as to eliminate introduction of artifact into the electrical signal being monitored, Henning et al., Col. 3, Lines 35-42.

In summary, in each of the electrochemical measurement techniques discussed above, accuracy (sensitivity and selectivity) is dependent upon the biosensor’ ability to detect an electrochemical phenomenon (e.g. flow of current, rise or drop in potential) that represents the characteristic signature (signal) of the of analyte of interest. Obviously, the accuracy of such measurement assumes that (a) the species responsible the electrochemical transition is unique within the analytical environment, and (b) the observed signal is solely attributable to species being measured. Unfortunately, biological fluid samples include a diverse chemical population of components that are endogenous to the sample and a number of components that are present because of exposure to or ingestion of exogenous agents. In each of the systems/protocols discussed above, relative to the electrochemical determination of glucose, the potential for interference is present, either because an interferent in the test sample undergoes an electrochemical transition similar to the analyte, or because such interferent inhibits measurement of the species that is being measured. Where limited success has been achieved, biosensor design and operation is not practical for economic manufacture or a self-testing environment. Thus, there continues to exist a need for development an electrochemical analysis system that is rugged (easily adapted to self-testing at home by the diabetic patient) and competitive with the cost of present glucose monitoring systems that utilize conventional spectrophotometric techniques.

### OBJECTS OF THE INVENTION

It is the object of this invention to remedy the above as well as related deficiencies in the prior art.

More specifically, it is the principle object of this invention to provide a system and method for the selective, electrochemical, quantitative measurement of an analyte in a aqueous sample by a transducer having an oxidase specific for the analyte of interest and an electrochemically active, metal doped carbon based electrode, specific for reduction of hydrogen peroxide.

It is yet another object of this invention to provide a system and method for the selective, electrochemical, quantitative measurement of glucose concentration in a aqueous sample utilizing a biosensor having an oxidase specific for the analyte of interest and an electrochemically active, metal doped carbon based electrode, specific for reduction of hydrogen peroxide.

It is still yet another object of this invention to provide a system and method for the direct, electrochemical, quantitative measurement of glucose concentration in a biological fluid sample utilizing a sensor comprising a transducer suitable for in-vitro or in-vivo detection of an analyte of interest.

Additional objects of this invention include a disposable test device for use in meter for the self-testing of glucose levels in biological fluids of diabetics.

### SUMMARY OF THE INVENTION

The above and related objects are achieved by providing a system and method for the selective analysis of a heterogeneous biological fluid sample for an analyte of interest, with a transducer comprising (a) an oxidase specific for the analyte of interest, and (b) a metal doped carbon composition specific for the catalytic reduction of hydrogen peroxide that has been liberated from the analyte of interest by the enzymatic action of said oxidase upon the analyte of interest.

In one of the preferred embodiments of this invention, the system utilizes a working electrode comprising an electrocatalytic, metal doped carbon based electrode specific for reduction of enzymatically-liberated hydrogen peroxide, and an oxidase specific for the analyte of interest. In another of the preferred embodiments, the working electrode includes a composition having both an electrocatalytic rhodium doped carbon and glucose oxidase.

The electrochemical analysis for the analyte of interest is accomplished by simply contacting a biological sample and a sensor assembly having an analyte recognition electrode and a counter electrode, or alternatively, an assembly having a analyte recognition-electrode, a counter electrode and a reference electrode. The contact of the sample with the sensor assembly, under analytical conditions, causes the oxidation of the analyte of interest by the oxidase, and the liberation of hydrogen peroxide, as a by-product of such oxidation. The electrocatalytic metal doped carbon composition converts the hydrogen peroxide (reduced) to water, with the corresponding liberation of free electrons at the working electrode. As free electrons are generated, current begins to flow (almost instantaneously) between the electrodes within the sensor assembly, reaching a relatively steady state, at a given potential. In the preferred embodiments of this invention, the potential within the cell is stepped over a given range (˜−0.3 to ˜+0.4 volts) to insure detection/observation of current fluctuations caused by the reduction of hydrogen peroxide.

In the preferred embodiments of this invention, the sensor assembly operation is compatible with both in vitro and in vivo analysis of the a biological fluid sample for glucose concentration in the management of a diabetics insulin and dietary needs.

The biosensors of this invention are relatively simple to fabricate and can be readily prepared from available materials, utilizing well-known assembly techniques. More specifically, the transducer of the working electrode of the biosensor is fabricated from a carbon based screen printing ink that is doped with a metal catalyst specific reduction of hydrogen peroxide. The preferred catalyst of the transducer of this invention is selected from among the group consisting of rhodium (Rh), ruthenium (Ru), iridium (Ir), their respective mixtures or an alloy thereof. The catalyst can also be introduced into the carbon matrix incident to the preparation of the microcarbon particles, or thereafter upon combination with the oxidase enzyme and a suitable binder. The consistency (viscosity) of the mixture is adjusted to permit screen printing of an electrode onto an insulating support layer. The preferred geometry of the screen printed material is in the form of an electrode strips, such as are illustrated in FIGS. 2 & 3..

The working electrode of the biosensor of this invention fabricated in the foregoing fashion has higher selectivity, higher sensitivity, and the greater versatility, than previously available, permitting its use in diverse environments and in analysis of heterogeneous biological fluids. These electrodes are also unique for their low background and rapid response in signal acquisition. The preferred biosensor of this invention comprises a two-electrode system; a working electrode and a counter electrode. Under some conditions, it may be desirable to add an additional reference electrode. The working electrode of this invention, unlike the dual enzyme based transducers disclosed in the prior art, does not suffer from the potential complexities in fabrication (e.g. matching of the enzyme substrate turnover of one enzyme to the turnover of the other), and, the potential inconsistency of results were such relationship is not maintained.

### BRIEF DESCRIPTION OF THE DRAWINGS

FIG. 1 is the reaction scheme illustrating the method of this invention wherein an analyte of interest (substrate) is enzymatically converted to hydrogen peroxide, which is, thereafter, catalytically reduced, under electrochemical conditions, so as to generate an electrical signal.

FIGS. 2A, 2B & 2C illustrate alternative biosensor configurations of this invention, specifically, a two electrode assembly (2A) having an analyte recognition sensor and a counter electrode, a three electrode assembly (2B) having an analyte recognition sensor, a reference electrode and a counter electrode, and sectional view of the biosensor the invention from one end thereof to the other (2C).

FIGS. 3A, 3B, 3C & 3D reveal, in greater detail, the components of each of the analyte recognition sensor configurations, upon disassembly of the sensor configurations illustrated in FIGS. 2A, 2B & 2C.

FIG. 4 provides a graphical comparison of hydrodynamic voltammograms for hydrogen peroxide at a rhodium doped carbon paste working electrode (A) and at a carbon paste working electrode devoid of rhodium (B).

FIG. 5 provides a graphical representation of the amperometric responses of a rhodium doped carbon paste working electrode to successive additions of hydrogen peroxide.

FIG. 6 provides a graphical representation of the amperometric responses of a rhodium doped carbon paste working electrode to successive additions of glucose (A) and the response of a carbon paste working electrode devoid of rhodium under similar conditions (B).

FIG. 7 provides a graphical of current-time responses of the rhodium-dispersed glucose biosensor to glucose (G), ascorbic acid (AA), acetaminophen (AC) and uric acid (UA).

FIG. 8 depicts current-time response curves for a ruthenium (Ru)—dispersed carbon screen printed sensor (see FIGS. 2 &3). Curve A depicts a current response for the biosensor over time relative to 50 mM, pH 7.4 PBS and curve B the current response over time relative to 30 mg/dL hydrogen peroxide.

FIG. 9 depicts current-time response curves for a copper (Cu)—dispersed carbon screen printed sensor (see FIGS. 2 &3). Curve A is a current response curve over time relative to 50 mM, pH 7.4 PBS and curve B the current response over time relative to 30 mg/dL hydrogen peroxide.

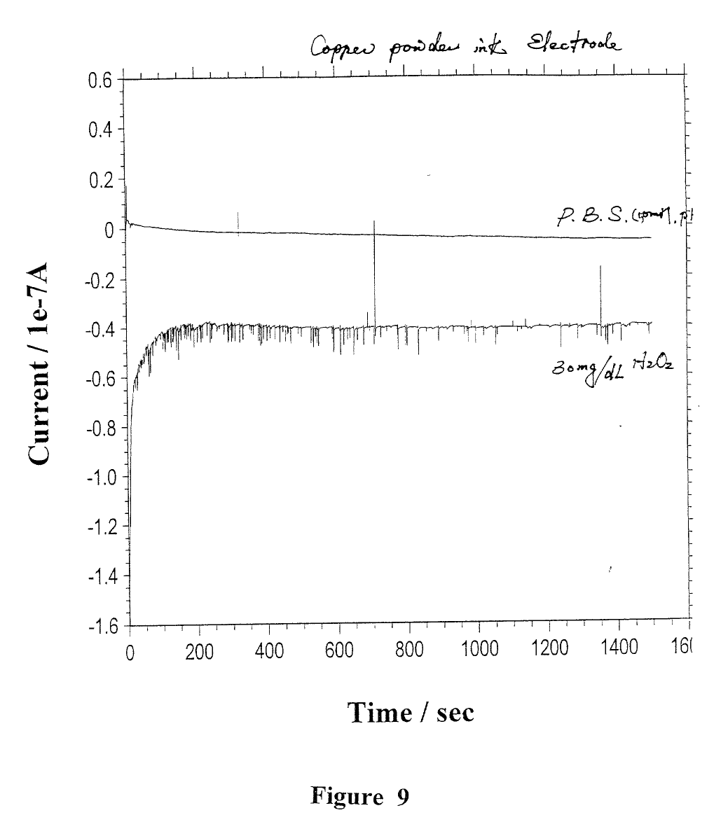

FIG. 10 depicts current response curves for glucose at a ruthenium (Ru)—dispersed carbon screen printed biosensor (see FIGS. 2 &3). Curve A is a current response curve over relative to a blank solution. Curve B is a current response over time relative to 100 nM glucose. Curve C is a current response curve over time relative to 10 mM glucose.

### DETAILED DESCRIPTION OF THE INVENTION INCLUDING PREFERRED EMBODIMENTS

The system and method of this invention are herein described in detail by reference to a model system for the selective electrochemical quantitative determination of glucose. In accordance with this invention, the biosensor is specifically configured for determination of a specific analyte of interest in a heterogeneous biological fluid sample. It is, thus, understood that the working electrode of the biosensor can include glucose oxidase, galactose oxidase, monoamine galactose oxidase, monoamine oxidase, L-amino acid oxidase, alcohol oxidase, xanthine oxidase, cholesterol oxidase, lactate oxidase and sarcosine oxidase, when the analyte of interest (substrate) is a glucose, galactose, monoamine, L-amino acid, alcohol, xanthine, cholesterol, lactate and sarcosine, respectively. Accordingly, the biosensors of this invention are efficacious for determination of glucose levels in diabetics, creatinine levels in individuals suffering from renal disease, cholesterol levels in individuals suffering from hypertension and blood alcohol levels in body fluids. Applications contemplated for the biosensors of this invention also include detecting lactate, acetylcholine, choline, hypoxanthine and xanthine.

In each instance, the biosensor is constructed from materials/components that are compatible with the contemplated analytical environment of the system and method of this invention. The conductive electrodes are preferably fabricated by screen printing of a conductive ink on an insulating support. The formulation of the conductive ink used for the working electrode comprises a microparticulate carbon having an average particle size of less than about 1000 nanometers (1 micron) in diameter, and preferably in the range of from about 5 to 50 nm in diameter. Such ink formulations are generally commercially available from a variety of sources. In the preferred embodiments of this invention, the carbon particle of the ink formulation are doped with effective amounts of metal catalyst specific for the reduction of hydrogen peroxide, or the metal catalyst combined with the ink formulation at the time of admixture with an oxidase enzyme. The fluid component of the ink formulation (preferably an aqueous or non-polar fluid) is compatible with both the oxidase enzyme and the subsequent test environment (to the extent any residual fluid is retained in the electrode coating/deposit. The preferred hydrogen peroxide reduction catalyst suitable for use in this invention is rhodium, ruthenium, iridium, their respective mixture or an alloy thereof.

The incorporation of a peroxide specific metal catalyst and an analyte specific enzyme (oxidase) directly into a coating on the working electrode enhance selectivity, sensitivity, and the versatility of the biosensor. Moreover, the proximity of the electrochemical reaction to the electrode surface decreases the background signal and increases electrode response to electrochemical changes in the electrode environment. These enhancements (sensitivity and selectivity) in the analysis of the analyte of interest in the heterogeneous biological sample is believed to be attributed to the electrocatalytic activity of the metal catalyst toward the reduction of enzymatically-liberated hydrogen peroxide. The electrodes utilized in the embodiments of the biosensors of this invention can be adapted for in-vitro or in-vivo detection of an analyte in biological specimens (e.g.: saliva, sweet, urine and serum) in other equally demanding environments.

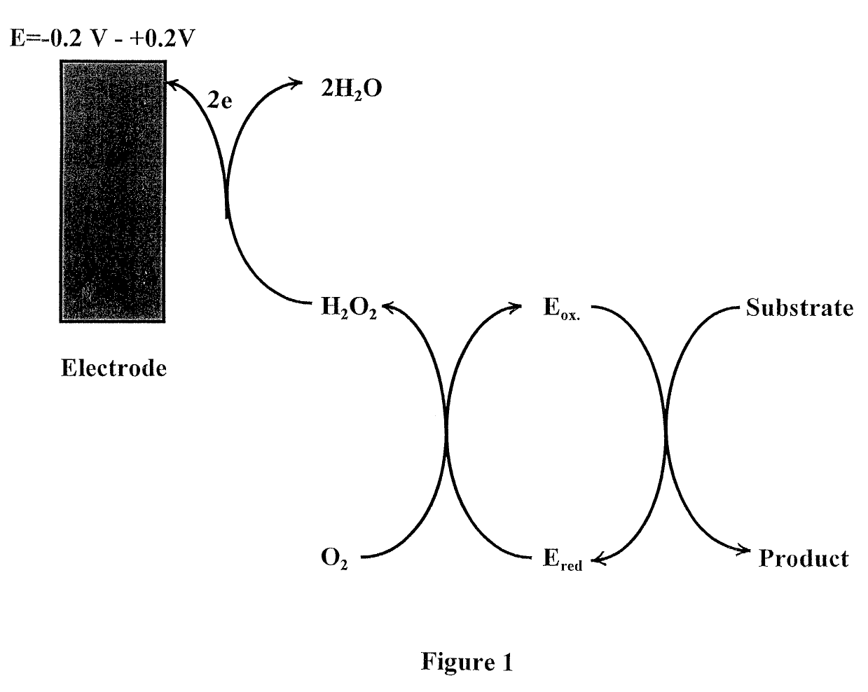

FIG. 1 illustrates the biochemical and electrochemical reaction cascade involved in the system and method of this invention. The reaction cascade, as it progresses from right to left, in this FIG. 1, involves the initial enzymatic oxidation of the analyte along with the liberation of hydrogen peroxide upon the contact of the an analyte specific enzyme (oxidase) with a test sample. In most instances, the analyte of interest is present at some basal or normal level, and the quantitative analysis thereof seeks to determine its presence above or below the basal or normal range. Where that analyte of interest is glucose, the normal or basal glucose level is typically within the range of from about 90-120 mgs/dl. Thus, quantitative analysis is critical to both the diagnosis and maintenance of the proper blood sugar balance. Accordingly, where interfering substances contribute to signal intensity, the value for the analyte of interest will be skewed (e.g. >150 mgs/dl), and thereby falsely indicate the need for medical intervention or an unnecessary insulin injection. Because of the specificity and sensitivity of the system and method of this invention, such analysis error is avoided and the test results can be relied upon for faithfiil and accurate reporting of critical analyte values, as a basis for medical decision making.

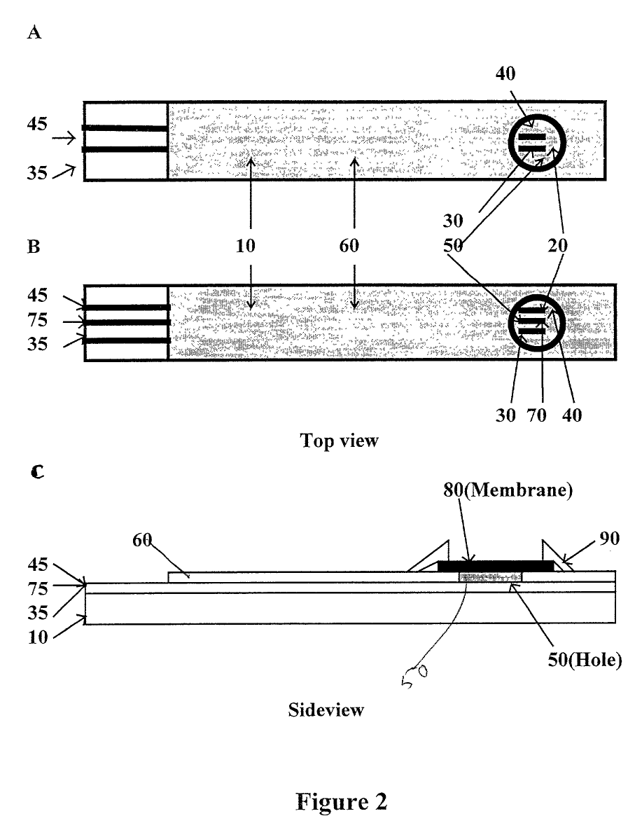

FIGS. 2 & 3 illustrate representative embodiments of the biosensors suitable for use in the system and method of this invention. FIG. 2A illustrates a biosensor configuration having a two-electrodes, a working electrode (30) and a counter electrode (40). FIG. 2B illustrates an alternative embodiment of the biosensor of this invention having three electrodes, a working electrode (30), a counter electrode (40) and a reference electrode (70). In each instance the operation thereof, relative to the system and method of the invention, is essentially the same. The additional electrode (70) in FIG. 2B simple provides a constant reference potential which maintains the potential of working electrode at the applied potential. The reference electrode (for three-electrode system) (70) is made by coating Ag/AgCl ink on the support (10). The electrode array is sandwiched between an insulating support (10) and insulating cover layer 60. The cover or top layer (60) is shorter in overall length that the insulating support so as to leave the electrode array exposed on the distal end of the biosensor for coupling to a source of power and a monitoring device. The proximal end of the top layer is provided with an aperture (50) to permit exposure and contact of the biological sample with the working electrode within the test area or site of the biosensor. In order to minimize particulate interference by the solids in the heterogeneous biological fluid sample, within the test area or site of the biosensor, a membrane filter (80) is place over the test site. Generally, the test site can range in diameter from about 0.5 to about 1 cm in diameter. The membrane filter (80) can be placed between the two insulating layers (90 & 60) of the sensor over the test site; or, affixed to the cover layer with a retainer (90) subsequent to the assembly of the biosensor and the isolation of the electrode array between the insulating layer (10) and cover layer (60) of the sensor.

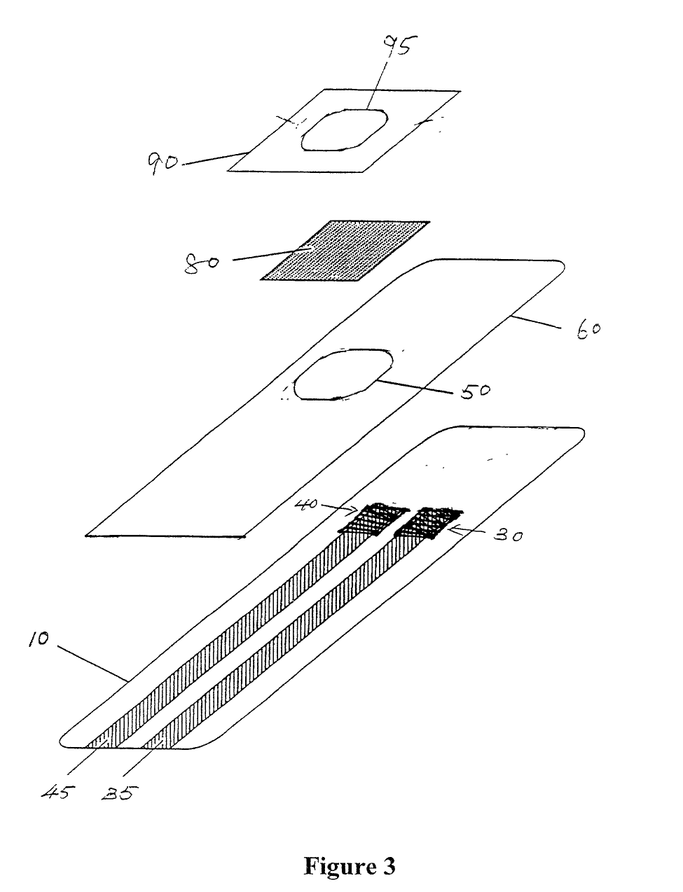

In FIG. 3, the biosensor configuration of FIG. 2C have been disassembled to illustrate the various components each such device. In FIG. 3, the insulating support layer (10) is shown with two electrodes (30, 40) running from the proximal end of the insulating layer to the distal end of the insulating layer (10). The cover layer (60) shown in FIG. 3 is somewhat shorter in length that the insulating layer (10) and is provided with an aperture (50) corresponding to the test site of the electrode array. The filter membrane (80), is larger in diameter than the test site aperture of the cover layer, and thereby of sufficient size to completely protect the aperture (50) of cover layer (60) from particulate matter. The retainer (90) for the filter membrane (80) is a water-proof material which is acted as to affixed to both the membrane (80) and to the cover layer (60) so as to retain the filter membrane in place over the aperture (50) of the cover layer. The retainer (90) is aligned with the aperture (50) so as to permit sample access to the test site through an coincident aperture (95) in the retainer. Once assembled the coincident features of the cover layer (60), filter membrane (80) and retainer (90) define a sample well over the test site of the biosensor. The filter membrane (80) is preferably cellulose acetate membrane or other biocompatable material. The membrane (80) can be used in its native state or treated with a substance to promote retention of particulates (coagulant for blood cells) on/within the membrane. The location of the aperture (50) in the cover layer (60), which is coincident with the test site of the electrode array, is determined by the relative placement of the working (30) and other electrodes (40, 70) on the support layer (10).

### EXAMPLES

The Examples which follow illustrate directly and by comparison, a number of the unique features of the biosenors used in the system and methods of this invention. Parts and percentages appearing such Examples are by weight, unless otherwise indicated. Apparatus and equipment used in the fabrication and evaluation of the biosensors set forth therein are also standard unless indicated to the contrary.

### Example 1

In order to compare and verify the selectivity of the working electrode of the biosensor of this invention, a series of experiments were devise to determine the electrochemical response of each of a rhodium doped carbon paste electrode, prepared in accordance with this invention, with the electrochemical response of a standard glucose oxidase-based electrode strip. In each case, each electrode was exposed to 2 mM hydrogen peroxide solution, the current monitored over a potential range of +0.4 to −0.3 volts and, recorded as a function of voltage.

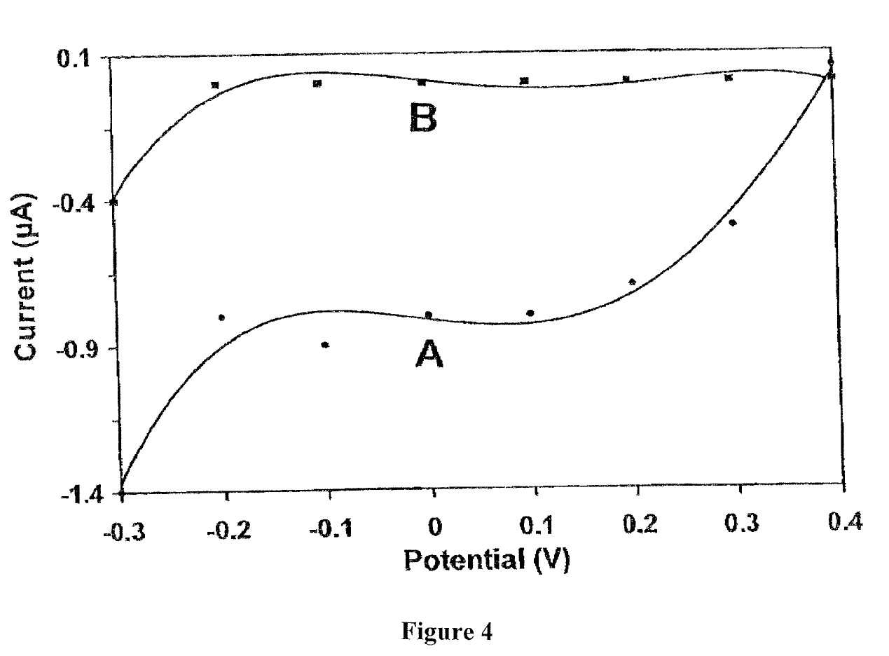

FIG. 4 graphically illustrates the results of such test. The conventional electrode strip (B), generated only a small anodic current at above +0.6 V (not shown) and no cathodic response was seen up to the potential −0.2 V. In comparison, the anodic sensing of the hydrogen peroxide for the rhodium-based electrode strip (A), showed generation of a discernable current starting at +0.4 V, while cathodic detection is feasible at more negative potentials. Accordingly, the range of potential for operation of the working electrode of an oxidase-based amperometric sensor in the system and method of this invention is relatively broad; and, the such working can operate effectively at an optimal potential region (+0.10 V to −0.20 V), where the interferent responses of common species can be minimized.

### Example 2

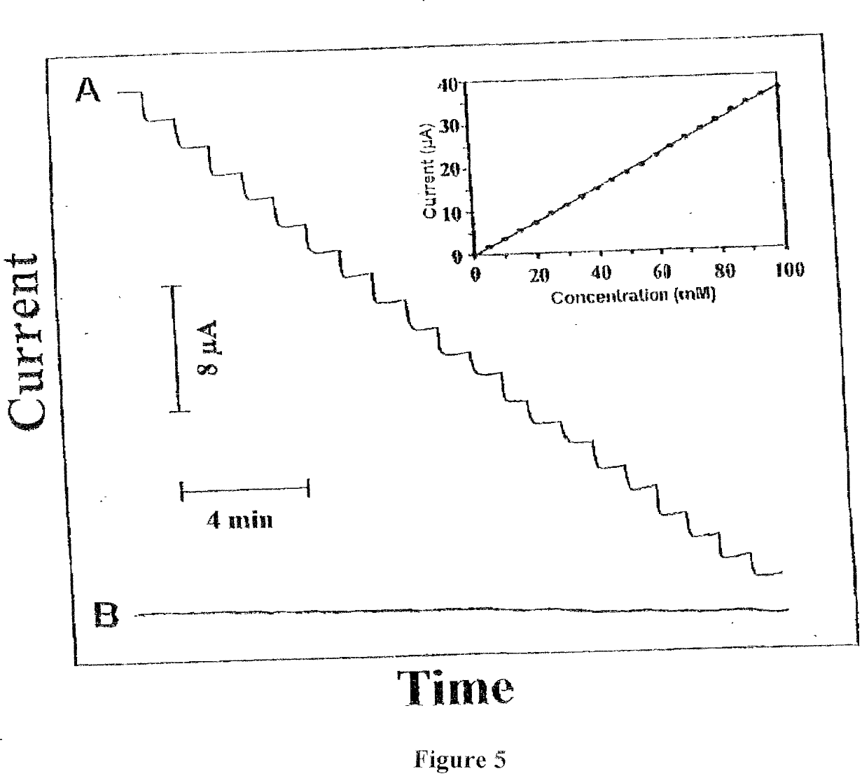

The procedures of Example 1 are repeated except that the current response is now measured at a constant potential of −0.05 V for successive additions of 5 mM hydrogen peroxide. FIG. 5 illustrates the current responses of rhodium-dispersed (A) and conventional (B) electrode strips measured over a four minute interval for each successive addition of 5 mM hydrogen peroxide. As expected, no signal response is observed with the conventional transducer (B), and a well-defined response is shown at rhodium-dispersed carbon paste transducer (A).

The significance of this observation confirms the selectivity of the working electrode of the system and method of this invention; specifically, the effective generation of current response at the working electrode to hydrogen peroxide over the range likely to be encountered in a diagnostic test environment at low potential. Thus, the ability to conduct such analysis at an electrical potential below required to generate an overlapping signal from common interference species.

### Example 3

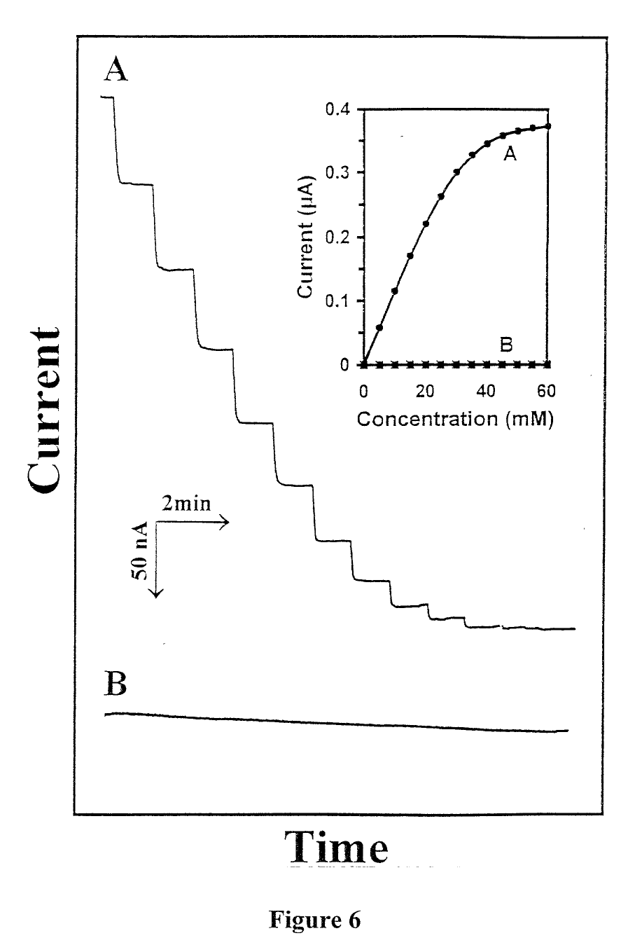

The procedures of Example 2 are repeated except that the working electrode comprises both a rhodium catalyst and glucose oxidase and the current response curve is measured at a −0.15 V. FIG. 6 illustrates the current responses of rhodium-dispersed (A) and conventional (B) electrode strips measured over a four minute interval for each successive addition of to successive additions of 5×10⁻³ M glucose. A well-defined current response for glucose is obtained at the metal-dispersed carbon enzyme-based transducer and no current signal for glucose is observed at the common carbon transducer in analogous measurements. The reaction occurring at the working electrode is very fast reaching a dynamic equilibrium, upon each addition of the sample solution, and generating a steady-state current signal within 20-30 sec. The linear response of metal-dispersed transducer to glucose is up to 30 mM of glucose. The signal response curve is effective at low detection limits for glucose because of favorable signal-to-noise ratio characteristics at −0.15 V.

### Example 4

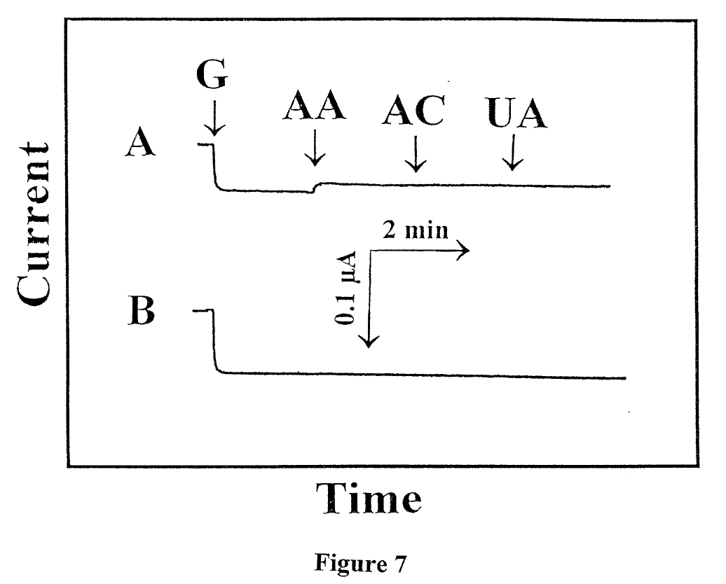

The procedures of Example 3 are repeated except that current response curve for glucose and various interferents (e.g. acetaminophen, uric and ascorbic acids, catecholamines) is measured over a range of from about +0.3 to −0.25V. FIG. 7 illustrates the current response of such compounds as a function of time. Cyclic voltammetric experiments indicated that the oxidation of the oxidizable species at the rhodium-dispersed carbon material starts at +0.20 V (ascorbic acid) and +0.30 V (acetaminophen, uric acid), with no reduction up to −0.25 V.

Curves were obtained by batch addition of interferences, ascorbic acid (AA), acetaminophen (AC) and uric acid (UA) after the glucose addition (G), at two different potentials (+0.20 (B) and −0.15 (C) V). A well-defined cathodic glucose response is obtained at the metal-dispersed carbon electrodes. At operating potential +0.20 V, such glucose response is accompanied by large anodic contributions from ascorbic and uric acids and acetaminophen. The use of lower operating potential greatly reduces these contributions. No interferences were observed at potential −0.10 V by a 10-fold increase of the concentration of the interference species (to 1 mM) indicating high selectivity towards the glucose substrate. Also, no interferences are found in untreated urine samples (to a 20-fold dilution in the cell solution). It is emphasized that such highly selective response to glucose is obtained at metaldispersed carbon electrodes without the use of mediators and permselective membranes.

### Example 5

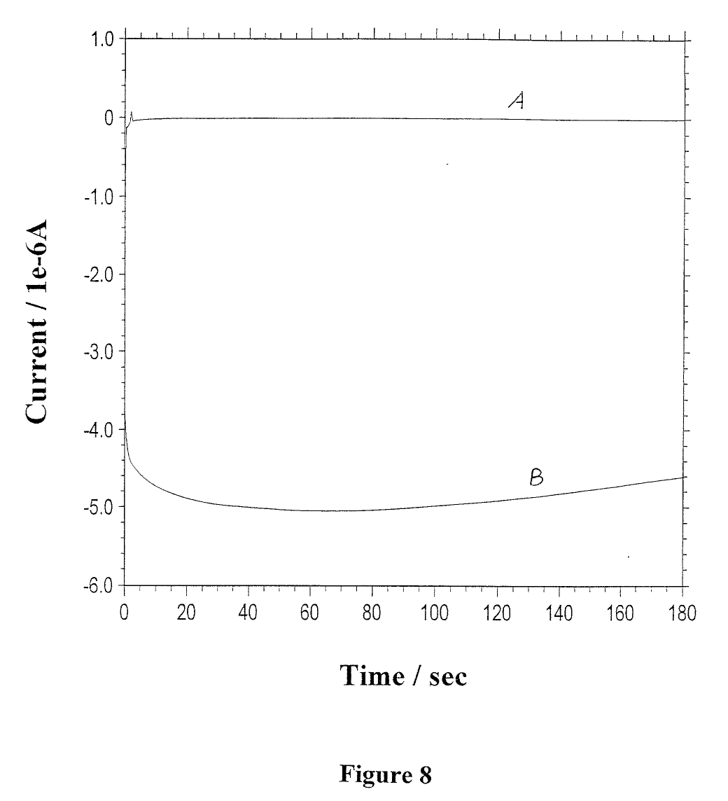

The screen printed biosensor made by catalytic metal doped carbon material is highly effective in its catalytic ability to reduce hydrogen peroxide, which reduction reaction can be effected at an electrode potential, near zero volt. The response curves of FIGS. 8 & 9 reflect the current responses over time at ruthenium and copper doped carbon screen printed electrode at applied potential, −0.05 V. Such metal doped carbon screen printed biosensor can be used to detect the glucose level in aqueous sample.

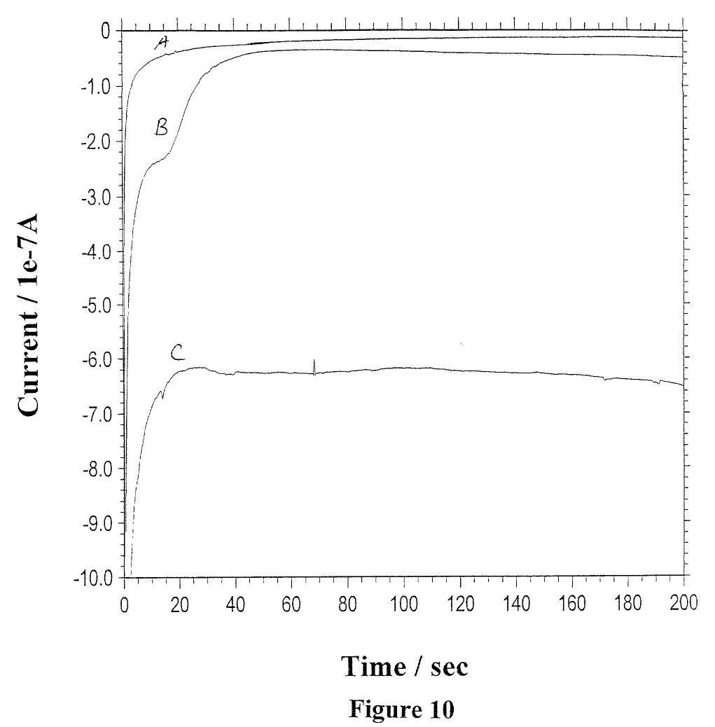

FIG. 10 also reflects the current responses of blank and glucose solutions at ruthenium doped carbon screen printed biosensor. Curve A represents the current response for a blank solution, curve B represents the current response for a 100 nM glucose solution and curve C is the represents the current response for a 10 mM glucose solution. The results depicted in this FIG. 10 confirms the rapid and stable current response of the screen printed biosensor of this invention a to glucose solution within 30 seconds. Because of its operation at near zero potential, the interferences in sample can be avoided.

The biosensors of the method and system of this invention are, thus, suitable for the highly selective in-vitro or in-vivo detection of numerous analytes in a variety of heterogeneous test samples (e.g.: saliva, sweet, urine serum and other specimen). Quantitative determinations of glucose in individuals suffering from diabetes, creatinine in individuals suffering from renal disease, cholesterol in individuals suffering from hypertension and alcohol in body fluids, are, thus, readily attainable in accordance with the system and method of this invention. Additional applications for the biosensor of this invention also include quantitative analysis of lactate, acetylcholine, choline, hypoxanthine and xanthine.

What is claimed is:

1. In a system for direct electrochemical measurement of enzymatically liberated hydrogen peroxide formed incident to the biocatalytic conversion of an analyte of interest, wherein said analyte is (a) initially exposed to an oxidase specific enzyme for biocatalytic conversion of said analyte into one or more by-products, including hydrogen peroxide, and (b) said hydrogen peroxide is reduced, under electrochemical conditions, so as to generate an electrical signal indicative of the presence and/or concentration of said analyte in said sample, wherein the improvement comprising:
A. Providing a biosensor assembly comprising a working electrode and a counter electrode, said working electrode having an effective amount of enzymatically active oxidase specific for biocatalytic conversion of an analyte of interest into one or more by-products, including hydrogen peroxide, and a metal doped carbon composition-specific for catalytic reduction of enymatically-liberated hydrogen peroxide;
B. Means for applying an electrical potential to said electrodes, wherein said potential is within a range of from about +0.4 to about −0.3;
C. Means for monitoring, under electrochemical conditions, the rate of catalytic reduction of enymatically-liberated hydrogen peroxide by said working electrode; and
D. Means for correlating said rate of reduction of said enymatically-liberated hydrogen peroxide by said working electrode with the concentration in said analyte in a aqueous fluid sample.

2. The improved system of claim 1, wherein the biosensor assembly comprises three electrodes, a working electrode, a counter electrode and a reference electrode.

3. The improved system of claim 1, wherein the a metal doped carbon composition comprises a hydrogen peroxide reduction effective amount of a metal catalyst selected from the group consisting of rhodium (Rh), ruthenium (Ru), iridium (Ir), their respective mixtures and an alloy thereof.

4. The improved system of claim 1, wherein the biosensor assembly is essentially devoid of a perm-selective membrane.

5. The improved system of claim 1, wherein in Step B said potential is within a range of from about +0.10 V to about −0.20 V

6. In a method for direct electrochemical measurement of enzymatically liberated hydrogen peroxide formed incident to the biocatalytic conversion of an analyte of interest, wherein said analyte is (a) initially exposed to an oxidase specific enzyme for biocatalytic conversion of said analyte into one or more by-products, including hydrogen peroxide, and (b) said hydrogen peroxide is reduced, under electrochemical conditions, so as to generate an electrical signal indicative of the presence and/or concentration of said analyte in said sample, wherein the improvement comprising:
A. Providing a biosensor assembly comprising an electrode array having a working electrode and a counter electrode, said working electrode having an effective amount of enzymatically active oxidase specific for biocatalytic conversion of an analyte of interest into one or more by-products, including hydrogen peroxide, and a metal doped carbon composition specific for catalytic reduction of enymatically-liberated hydrogen peroxide;
B. Applying an aqueous test sample suspected of containing an analyte of interest to said electrode array of said biosensor, under biocatalytic conditions;
C. Applying an electrical potential to said electrode array under catalytic reduction conditions;
D. Monitoring an electrical signal generated by said catalytic reduction of enymatically-liberated hydrogen peroxide over a period of time until constant;
E. Correlating said constant electrical signal with a concentration of said analyte in said aqueous sample.

7. In a biosensor assembly for the selective determination of an analyte in an aqueous sample by direct electrochemical measurement of enzymatically liberated hydrogen peroxide, the improvement comprising:
a working electrode and a counter electrode, said working electrode comprising an electrically conductive deposit having an effective amount of enzymatically active oxidase specific for biocatalytic conversion of an analyte of interest into one or more by-products, including hydrogen peroxide, and a metal doped carbon composition specific for catalytic reduction of enymatically-liberated hydrogen peroxide, said working electrode being devoid of a perm-selective membrane and yet effective for selective determination of enymatically-liberated hydrogen peroxide over a potential range of about +0.10 V to about −0.20 V in an aqueous sample.

8. The improved biosensor of claim 7, wherein said electrodes are sandwiched between a first insulating layer and a second insulating layer, said first insulating layer being longer than said insulation layer, and said second insulating layer having an aperture corresponding to an analysis or test site for application of an aqueous test sample onto said working electrode.

9. The improved biosensor of claim 8, wherein the test site includes a filter member for particulate materials contained in said aqueous sample.

10. The improved biosensor of claim 8, wherein the biosensor assembly comprises three electrodes, a working electrode, a counter electrode and a reference electrode.

11. The improved biosensor of claim 8, wherein the working comprises a hydrogen peroxide reduction effective amount of a rhodium catalyst.
